(19) United States
(12) Patent Application Publication
(10) Pub. No.: US 2025/0058075 A1
(43) Pub. Date: Feb. 20, 2025

(54) Method and System for Treating Depression and Other Mental Disorders

(71) Applicant: SNOWFALL PSYCHEDELICS LLC, Liberty Hill, TX (US)

(72) Inventor: Adam Slank, Liberty Hill, TX (US)

(73) Assignee: SNOWFALL GROUP LLC, Liberty Hill, TX (US)

(21) Appl. No.: 18/235,036

(22) Filed: Aug. 17, 2023

(51) Int. Cl. A61M 21/02; A61K 31/357; A61K 31/675

(52) U.S. Cl. CPC A61M 21/02; A61K 31/357; A61K 31/675; A61M 2021/0027; A61M 2021/0077

(57) ABSTRACT

According to an embodiment, the present invention provides a method for treating DEPRESSION, comprising administering to a subject in need thereof a therapeutically effective amount of psilocybin and MDMA. This combination aims to enhance the positive psychological effects of the therapy, creating a beneficial environment for the treatment of DEPRESSION.

### TECHNICAL FIELD

The present disclosure is directed towards methods and systems for treating depression and other mental disorders.

### BACKGROUND OF THE DISCLOSURE

Depression is a debilitating mental health condition leading to a range of symptoms including apathy, low self-esteem, anxiety, and uncontrollable thoughts. Current treatments for depression mainly consist of psychotherapy, cognitive behavioral therapy, and pharmacotherapy including selective serotonin reuptake inhibitors (SSRIs) and serotonin and norepinephrine reuptake inhibitors (SNRIs). However, not all individuals respond sufficiently to these treatments, indicating a need for novel therapeutic approaches.

There exists a need in the art to address the problems described above.

### SUMMARY OF THE DISCLOSURE

The present disclosure discloses methods directed towards treating depression and other mental disorders and increasing the favorable therapeutic outcomes associated with the use of oral compositions.

According to an embodiment, the present invention provides a method for treating depression and other mental disorders such as PTSD, comprising administering to a subject in need thereof a therapeutically effective amount of psilocybin and MDMA and/or MDA. Treatment using the systems and methods disclosed herein may also be for: bipolar disorder, obsessive compulsive disorder, alcohol use disorder, treatment resistant PTSD.

This combination aims to enhance the positive psychological effects of the therapy, creating a beneficial environment for the treatment of depression and other mental disorders. According to embodiments of the present disclosure, a favorable outcome is achieved by triggering a positive psychological condition in a person through the administration of an MDMA-based empathogen/entactogen, in a dosage ranging from 10 to 300 milligrams.

In alternative embodiments, wherever the use of psilocybin is mentioned in the present disclosure, it may be understood that psilocin could be substituted in equivalent doses. This is because psilocin, like psilocybin, is an active compound found in psychedelic mushrooms and shares a similar molecular structure. The body metabolizes psilocybin into psilocin. Therefore, the direct use of psilocin could theoretically provide the same therapeutic benefits. The equivalence in dosage between the two substances should be calculated considering the fact that psilocin is the active metabolite, and psilocybin's weight includes an extra phosphate group which is lost during metabolism. Any therapeutic regimen, interactions, contraindications, and side effects mentioned for psilocybin in this application would therefore apply equivalently to psilocin.

Additionally, according to further embodiments extracts derived from psilocybin can also be utilized within the parameters of this application in place of psilocybin itself. The use of such extracts allows for precise dosing and control, thus potentially minimizing side effects while maximizing therapeutic benefits. Furthermore, these extracts can be processed to ensure the exclusion of other compounds in the mushroom which may cause undesired effects. According to embodiments of the disclosure, the therapeutic regimen, interactions, contraindications, and side effects outlined for psilocybin in this application would extend to its extracts as well. The dosage equivalency would need to be calculated to reflect the potency of the extracts.

Although specific advantages have been enumerated above, various embodiments may include some, none, or all of the enumerated advantages. Additionally, other technical advantages may become readily apparent to one of ordinary skill in the art after review of the following figures and description.

### BRIEF DESCRIPTION OF THE DRAWINGS

For a more complete understanding of the present disclosure and its advantages, reference is now made to the following description taken in conjunction with the accompanying drawings, in which like reference numerals represent like parts.

FIG. 1 illustrates an example process according to an embodiment of the present disclosure.

### DETAILED DESCRIPTION OF THE DISCLOSURE

It should be understood at the outset that, although exemplary embodiments are illustrated in the figures and described below, the principles of the present disclosure may be implemented using other techniques. The present disclosure should in no way be explicitly limited to the exemplary implementations and techniques illustrated in the drawings and described below. Additionally, unless otherwise specifically noted, articles depicted in the drawings are not necessarily drawn to scale

The present disclosure is directed towards method and compositions intended to enhance the immediate and qualitative positive improvement of the subjective effects, including emotional and therapeutic, of psychedelic substances used in medical treatment, primarily for psychiatric disorders.

The present disclosure presents a method that amplifies both the immediate and lasting therapeutic benefits of a psychedelic by stimulating a positive psychological state in a subject, and subsequently boosting the positive response to the psychedelic. An objective of this invention is to optimize the positive acute subjective effects in response to a primary therapeutic agent, while diminishing the adverse effects, leading to a higher ratio of beneficial over negative responses to the composition.

According to one aspect of the present invention, the method includes the administration of psilocybin as the primary therapeutic agent, a naturally occurring psychedelic compound produced by more than 200 species of fungi, known for its mind-altering effects. According to an embodiment, the psilocybin is administered in a dosage of between 10-500 mg.

In another aspect of the invention, the method involves the administration of MDMA as the primary therapeutic agent, a synthetic drug that alters mood and perception, inducing feelings of increased energy, pleasure, emotional warmth, and distorted sensory and time perception. According to an embodiment, the MDMA is administered in a dosage of between 20-300 mg. In another aspect of the present disclosure, the method involves the administration of MDA in a dosage between 20-300 mg.

According to an embodiment, the psilocybin and the MDMA may be administered concurrently in the primary therapeutic agent. According to a second embodiment, the MDMA may be administered prior to the administration of psilocybin. Both substances can be administered orally, in forms including but not limited to tablets, capsules, or liquid solutions/suspensions. According to an embodiment, the MDMA is administered prior to administration of psilocybin by using a time-controlled capsule.

A further embodiment includes providing a kit for treating depression, with a primary therapeutic agent which includes psilocybin and MDMA, as well as instructions for their administration to a subject. According to a further embodiment, the composition further includes adaptogenic and pro-serotonin supplements to the blend (such as 5HTP) that aid the transition and recovery after a treatment. The aim is to equip patients and caregivers with an easy-to-follow guide to using the medication safely and effectively. According to an embodiment the kit is provided with a set of clear, simple, and user-friendly instructions for administering these potent therapeutic agents. The aim of this instruction manual is to empower patients and their caregivers by providing them with a step-by-step guide for safe and effective usage of the medication, detailing the correct dosages, the timing of administration, potential side effects, and ways to manage them.

In yet another embodiment of the present invention, the therapy is delivered via a specially designed time-release capsule. The capsule is engineered to first release the primary therapeutic agent, (such as MDMA and/or MDA together with psilocybin), into the user's system, initiating its psychedelic effects. This initial release is calculated to maximize therapeutic effectiveness and ensure a manageable and safe psychedelic experience. Following the controlled release of the MDMA, the capsule is designed to subsequently release a pro-serotonin compound. This second release aims to replenish the serotonin levels in the user's brain, counteracting potential comedown effects from the MDMA and promoting a smoother transition back to normal states of consciousness. The timing and dosage of the pro-serotonin compound are calibrated to correspond with the MDMA's metabolism, thus ensuring an optimal balance of neurochemical activity and further enhancing the overall safety and efficacy of the treatment.

According to an embodiment, pharmaceutical composition is specially formulated for oral administration, thus providing a convenient method for those seeking treatment. This non-invasive approach not only simplifies the delivery mechanism but also increases the likelihood of consistent usage and, therefore, successful treatment.

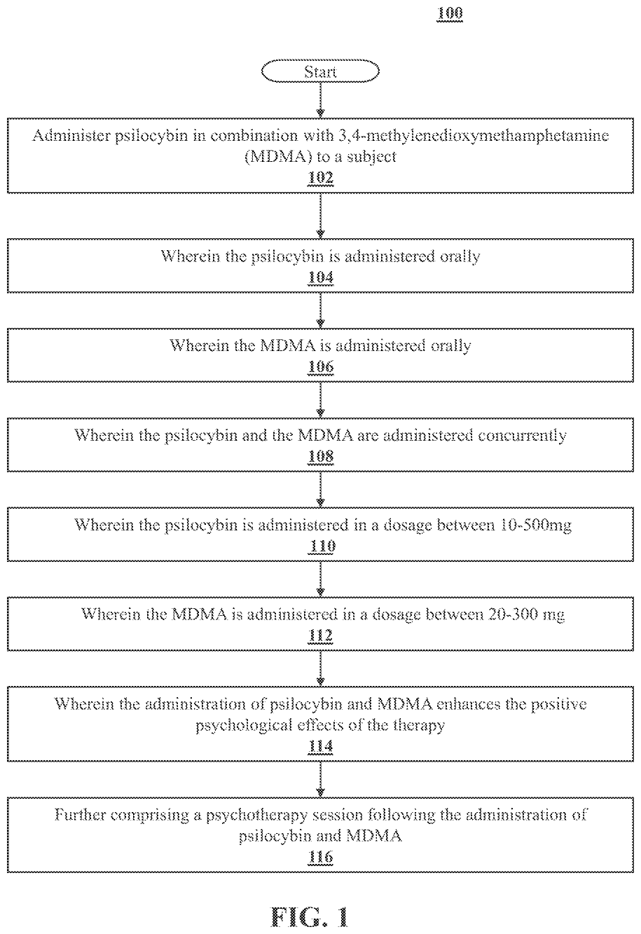

FIG. 1 is a flowchart of an example process 100. In some implementations, one or more process blocks of FIG. 1 may be performed by a patient.

As shown in FIG. 1, process 100 may include administering psilocybin in combination with 3,4-methylenedioxymethamphetamine (MDMA) to a subject (block 102). For example, patient may administer psilocybin in combination with 3,4-methylenedioxymethamphetamine (mdma) to a subject, as described above.

Process 100 may include additional implementations, such as any single implementation or any combination of implementations described below and/or in connection with one or more other processes described elsewhere herein. In a first implementation, the psilocybin is administered orally (block 104).

In a second implementation, alone or in combination with the first implementation, the MDMA is administered orally (block 106).

In a third implementation, alone or in combination with the first and second implementation, the psilocybin and the MDMA are administered concurrently (block 108).

In a fourth implementation, alone or in combination with one or more of the first through third implementations, the MDMA is administered prior to the administration of psilocybin.

In a fifth implementation, alone or in combination with one or more of the first through fourth implementations, the psilocybin is administered in a dosage between 10-500 mg (block 110).

In a sixth implementation, alone or in combination with one or more of the first through fifth implementations, the MDMA is administered in a dosage between 20-300 mg (block 112).

In a seventh implementation, alone or in combination with one or more of the first through sixth implementations, the administration of psilocybin and MDMA enhances the positive psychological effects of the therapy (block 114).

An eighth implementation, alone or in combination with one or more of the first through seventh implementations, process 100 may include a psychotherapy session following the administration of psilocybin and MDMA (block 116).

Although FIG. 1 shows example blocks of process 100, in some implementations, process 100 may include additional blocks, fewer blocks, different blocks, or differently arranged blocks than those depicted in FIG. 1. Additionally, or alternatively, two or more of the blocks of process 100 may be performed in parallel.

According to an embodiment, to enhance the efficacy of the treatment, the present invention also includes the addition of adaptogenic and pro-serotonin supplements to the primary therapeutic agent. Adaptogens, which include a variety of non-toxic plants such as Asian ginseng, ashwagandha, lion's mane, and Rhodiola rosea, are believed to help the body resist various stressors. Similarly, pro-serotonin supplements, such as 5HTP, are intended to boost serotonin levels, which can further contribute to alleviating symptoms of depression. The synergistic effect of these components potentially increases the overall effectiveness of the depression treatment, providing an innovative and comprehensive approach to addressing this significant mental health issue. Embodiments of the present disclosure may include pineapple and pineapple extracts and powders (a naturally occurring source of tryptophan) as the pro-serotonin supplement. Additional embodiments may include tryptophan itself in pure form as the pro-seretonin supplement.

Furthermore, embodiments of the present invention contemplate multiple delivery methods for the administration of psilocybin and MDMA and/or MDA. While oral administration in the form of tablets, capsules, or liquid solutions/suspensions is common, other embodiments could also be utilized to cater to the needs of different individuals or clinical scenarios.

In one embodiment, psilocybin and MDMA, along with possible additional components, may be incorporated into a sublingual strip. Such strips, designed to dissolve quickly when placed under the tongue, allow for rapid absorption of the active compounds directly into the bloodstream through the mucous membranes. This method can facilitate quicker onset of the effects, bypassing the digestive system, which may be advantageous in certain therapeutic contexts.

Alternatively, the administration of the pharmaceutical composition may be achieved through the use of an inhaler. In this form, the active compounds are delivered directly to the lungs, where they can be quickly absorbed into the bloodstream. This method provides another route for swift onset of the effects and can be particularly useful for individuals who may have difficulty with oral administration.

Moreover, a solution form of the pharmaceutical composition can be formulated for oral consumption, intravenous injection, or even as a nasal spray. Solutions offer flexibility in terms of dosage adjustments and can be a preferred delivery method for some patients or in specific therapeutic settings.

Each of these delivery methods—sublingual strips, inhalers, and solutions—expand the versatility and applicability of the therapeutic combination of psilocybin and MDMA, enabling the tailoring of depression treatment to the unique needs and preferences of each individual patient.

The present invention also provides potential dose ranges for the aforementioned therapeutic components. The composition may include a therapeutically effective amount of psilocybin, which could be, for example, in the range of about 10 to 25 milligrams per dose. This range is based on psilocybin's therapeutic window as observed in clinical studies, taking into account individual variation and tolerability.

For the second main component, MDMA, a therapeutically effective amount could range from about 75 to 200 milligrams per dose. These suggested ranges are informed by the compound's efficacy in the context of depression treatment, as demonstrated in various clinical trials, and are subject to individual therapeutic response and safety considerations.

As for the adaptogenic components of the composition, the dosage may vary depending on the specific adaptogen utilized. For example, a therapeutically effective dose of ashwagandha might be in the range of about 300 to 500 milligrams per day. Similarly, Rhodiola rosea may be included in the amount of about 100 to 300 milligrams per dose.

With respect to the pro-serotonin supplement, such as 5HTP, the dosage might range from about 50 to 100 milligrams per day. This is based on the compound's established safe and effective range for increasing serotonin levels.

These dosage ranges are merely illustrative and the actual doses may be adjusted by a

healthcare provider based on several factors, including but not limited to the patient's age, weight, sex, medical history, and severity of depression symptoms. It is intended that these dosage ranges provide a practical guideline for physicians and other medical professionals when prescribing this novel therapeutic composition for the treatment of depression.

In one embodiment of the present invention, the method comprises the provision of an interactive mobile application. The application delivers voice prompts, offering a structured treatment plan. The pre-treatment phase includes prompts to help the user prepare for the experience, including environmental setup, reminders for hydration and nutrition, and mindfulness exercises. During the therapeutic administration, the application provides soothing, therapeutic auditory guidance that complements the effects of the therapeutic composition. It may include suggestions to focus on breath, positive affirmations, reminders about the transitory nature of any potential discomfort, and prompts to encourage introspection or reflection on specific therapeutic themes. The post-treatment phase includes voice prompts to guide the user in reflecting upon and integrating the experiences they had during the treatment. Additionally, the application includes safety features to allow the user to seek help if needed and monitors the user's well-being through mood tracking and self-report surveys.

In accordance with an embodiment the application provides voice prompts to guide the user throughout the process, including the preparatory phase, the active journey phase, and the integration phase after the journey. This method enables self-administration of the medicine at home or in an unsupervised setting, thereby increasing accessibility and patient comfort.

In accordance with an embodiment mobile application extends its functionality by not only providing voice prompts but also incorporating therapeutic music. The voice prompts guide the user during preparation, active journey, and post-journey integration phases, while the music serves to enhance the therapeutic process and user experience. The combination of auditory guidance and musical therapy optimizes the potential of the MDMA treatment in an at-home or unsupervised setting.

In a further embodiment the mobile application is designed to provide a solely music-based guidance system, eliminating the use of voice prompts. Instead, the application features a selection of therapeutic music tracks that the user can play during the preparatory phase, the journey, and the post-journey integration phase. This unique combination of medicine and music therapy can be particularly beneficial for users who respond more positively to music stimuli, creating a personalized and soothing therapeutic experience.

In a further embodiment the mobile application focuses solely on voice-guided prompts. The voice prompts offer detailed guidance for the user throughout the preparatory stage, the active journey, and the post-journey integration phase. By providing a consistent, verbal guide without the addition of music, this method supports users who prefer a focused, language-based approach to navigate their therapeutic experience. This voice guidance system is designed to be used at home or in an unsupervised setting.

In another embodiment of the present invention, the therapeutic composition medicine can be administered under a physician's supervision or prescribed for at-home use by the patient. The application will guide the user on correct dosage and administration timing, as well as precautionary measures, ensuring optimal safety. The combination of the application and the therapeutic composition is designed to reduce the barriers to effective mental health treatment, enhance the therapeutic process, and help users manage their mental health in a safe and self-directed manner. As such, this invention has the potential to transform mental health care by offering a safe, effective, and user-friendly approach to managing depression and other related disorders.

This method can be utilized for any indications where psychedelic medications are employed and is generally pertinent to situations where a positive post-treatment experience with psychedelics predicts long-term outcomes. This typically includes, but is not restricted to, psychiatric disorders such as depression, PTSD, anxiety, life-threatening disease-related anxiety, obsessive-compulsive disorder, personality disorders, and addiction.

Modifications, additions, or omissions may be made to the systems, apparatuses, and/or methods described herein without departing from the scope of the disclosure. For example, various components of the systems and apparatuses may be integrated or separated. Moreover, the operations of the systems and apparatuses disclosed herein may be performed by more, fewer, or other components and the methods described may include more, fewer, or other steps. Additionally, steps may be performed in any suitable order. As used in this document, “each” refers to each member of a set or each member of a subset of a set.

To aid the Patent Office and any readers of any patent issued on this application in interpreting the claims appended hereto, applicants wish to note that they do not intend any of the appended claims or claim elements to invoke 35 U.S.C. § 112 (f) unless the words “means for” or “step for” are explicitly used in the particular claim.

1. A method for treating post-traumatic stress disorder the method comprising: administering a primary therapeutic agent comprising psilocybin in combination with 3,4-methylenedioxymethamphetamine to a subject.

2. The method of claim 1, wherein the psilocybin is administered orally.

3. The method of claim 2, wherein the MDMA is administered orally.

4. The method of claim 3, wherein the psilocybin and the MDMA are administered concurrently.

5. The method of claim 3, wherein the MDMA is administered prior to administration of psilocybin by using a time-controlled capsule.

6. The method of claim 5, wherein the psilocybin is administered in a dosage between 10-500 mg.

7. The method of claim 6, wherein the MDMA is administered in a dosage between 20-300 mg.

8. The method of claim 7, further comprising a psychotherapy session following the administration of psilocybin and MDMA.

9. The method of claim 8, wherein the primary therapeutic agent is administered in conjunction with a software application that provides voice prompts to guide the subject through a preparation stage, a psychedelic journey, and a post-journey integration stage.

10. The method of claim 9, wherein the software application further comprises a mobile application providing guidance includes not only voice prompts for preparation, during the journey, and post-journey integration, but also features therapeutic music, all combined to create a comprehensive at-home or unsupervised treatment experience.

11. The method of claim 10, wherein the mobile application substitutes the voice prompts with a selection of therapeutic music tracks, providing a solely music-based guidance system for the user during the preparation stage, the journey, and the post-journey integration phase.

12. The method of claim 11, wherein the mobile application is designed to provide only voice-guided prompts for preparation, during the journey, and post-journey integration, offering a focused, verbal guidance-based therapeutic experience for at-home or unsupervised use.

13. A pharmaceutical composition for treating depression, comprising a therapeutically effective amount of psilocybin and MDMA, wherein the composition is formulated for oral administration.

14. The pharmaceutical composition of claim 13, wherein the composition is in the form of a tablet.

15. The pharmaceutical composition of claim 14, wherein the composition is in the form of a liquid solution.

16. A kit for treating depression, comprising: a first component including psilocybin; a second component including MDMA; and instructions for administering the psilocybin and MDMA to a subject.

17. The kit of claim 16, wherein the instructions direct that the MDMA is to be administered prior to the administration of psilocybin.

18. The kit of claim 16, wherein the instructions direct that the psilocybin and the MDMA are to be administered concurrently.
